(19) United States
(12) Patent Application Publication
(10) Pub. No.: US 2025/0281123 A1
(43) Pub. Date: Sep. 11, 2025

(54) TRANSCATHETER HEART VALVE WITH DEFORMABLE INDUCTOR FOR DUAL WIRELESS PRESSURE MONITORING

(71) Applicant: EDWARDS LIFESCIENCES CORPORATION, Irvine, CA (US)

(72) Inventors: Rendle Lamarr Myles, JR., Covina, CA (US); Laxmi Ray, Costa Mesa, CA (US); Blake W. Axelrod, South Pasadena, CA (US)

(73) Assignee: EDWARDS LIFESCIENCES CORPORATION, Irvine, CA (US)

(21) Appl. No.: 19/218,003

(22) Filed: May 23, 2025

Related U.S. Application Data
(63) Continuation of application No. PCT/US2023/084312, filed on Dec. 15, 2023.
(60) Provisional application No. 63/387,915, filed on Dec. 16, 2022.

(51) Int. Cl. A61B 5/00; A61B 5/0215; A61B 5/07; A61F 2/24; A61L 31/02

(52) U.S. Cl. CPC A61B 5/6862; A61B 5/0215; A61B 5/076; A61F 2/2418; A61L 31/022; A61L 2430/20

(57) ABSTRACT

A prosthetic valve includes a flexible frame disposed along and deformable about a frame axis. The frame includes a first end, a second end oppositely disposed from the first end, and a network of interconnected struts defining a plurality of cells. A first circuit is mounted on the frame. The first circuit includes a first inductor coil attached to and tracing a first subset of the struts such that the first inductor coil outlines a first subset of the plurality of cells, and a first sensor in electrical communication with the first inductor coil. The first sensor is configured to sense a physical parameter and generate a signal representing the physical parameter.

### CROSS-REFERENCE TO RELATED APPLICATION(S)

This application is a continuation of International Application No. PCT/US2023/084312, filed Dec. 15, 2023, which claims the benefit of U.S. Provisional Application No. 63/387,915, filed Dec. 16, 2022, the disclosures of which are hereby incorporated by reference in their entireties.

### BACKGROUND

The present disclosure relates to medical implant devices, and in particular, to implantable prosthetic valves.

Patients who receive heart valve implants can suffer from post-operation complications. Risk of complications is especially high within thirty or sixty days of an implant operation. However, during such periods of time, the patient may no longer be in a hospital or extended care facility/system, and therefore complications that arise may require reentry into the care system, potentially adding significant cost to the overall patient treatment. Furthermore, increased health risks may result from the patient delaying return to the hospital due to failure to recognize the complications until they manifest through perceivable symptoms that the patient interprets as requiring hospital care. Accordingly, systems, devices and methods for post-operatively monitoring prosthetic heart valve implant recipients, including in an environment outside of the hospital or care facility, are desirable for improving patient outcomes

### SUMMARY

In one example, a prosthetic valve includes a flexible frame disposed along and deformable about a frame axis. The frame includes a first end, a second end oppositely disposed from the first end, and a network of interconnected struts defining a plurality of cells. A first circuit is mounted on the frame. The first circuit includes a first inductor coil attached to and tracing a first subset of the struts such that the first inductor coil outlines a first subset of the plurality of cells, and a first sensor in electrical communication with the first inductor coil. The first sensor is configured to sense a physical parameter and generate a signal representing the physical parameter.

In another example, a prosthetic valve assembly includes a prosthetic valve with a flexible frame disposed along a frame axis and deformable about the frame axis between a crimped state and an expanded state. The frame includes a first end, a second end oppositely disposed from the first end, and a network of interconnected struts defining a plurality of cells. A first circuit is mounted on the frame. The first circuit includes a first inductor coil attached to and tracing a first subset of the struts such that the first inductor coil outlines a first subset of the plurality of cells, and a first sensor in electrical communication with the first inductor coil. The first sensor is configured to sense a physical parameter and generate a signal representing the physical parameter. The prosthetic valve assembly further includes a transmitter in communication with the first sensor.

In another example, a prosthetic valve includes a flexible frame disposed along a frame axis and deformable about the frame axis between a crimped state and an expanded state. The frame includes a first end, a second end oppositely disposed from the first end, and a network of interconnected struts defining a plurality of cells. A multilayer sensing assembly is mounted on the frame. The multilayer sensing assembly includes a first inductor coil pair comprising first upper and lower inductor coil portions, the first inductor coil pair disposed on a flexible substrate, a detuning mitigation layer disposed between the frame and the flexible substrate, and a first sensor in electrical communication with the first inductor coil pair. The first sensor is configured to sense a physical parameter and generate a signal representing the physical parameter.

### BRIEF DESCRIPTION OF THE DRAWINGS

FIG. 1 is a schematic illustration of a human patient with a heart.

FIG. 2 is a partial cross-sectional schematic of the heart.

FIG. 3 is a block diagram representing a monitoring system for monitoring one or

more physiological parameters associated with a patient.

FIG. 4 is a perspective view of a first example of a prosthetic heart valve shown in an expanded state.

FIG. 5 is a front view of a sensing circuit of the prosthetic heart valve of FIG. 4, shown in isolation from the frame.

FIG. 6 is a schematic cross-sectional exploded view of a strut and an inductor coil of the prosthetic heart valve of FIG. 4 taken transverse to axis A of FIG. 4.

FIG. 7 is a perspective view of a second example of a prosthetic heart valve shown in the expanded state.

FIG. 8 is a schematic front view of a third example of a prosthetic heart valve shown in a crimped state.

FIG. 9 is a schematic plan view comparing radial dimensions of a frame of the third example of the prosthetic heart valve in the expanded state and the crimped state.

FIG. 10 is a schematic view of a fourth example of a prosthetic heart valve shown in the expanded state with a fabric cover over the inductor coil.

FIG. 11 is a schematic cross-sectional view showing a portion a multilayer sensing assembly disposed on a flexible frame.

### DETAILED DESCRIPTION

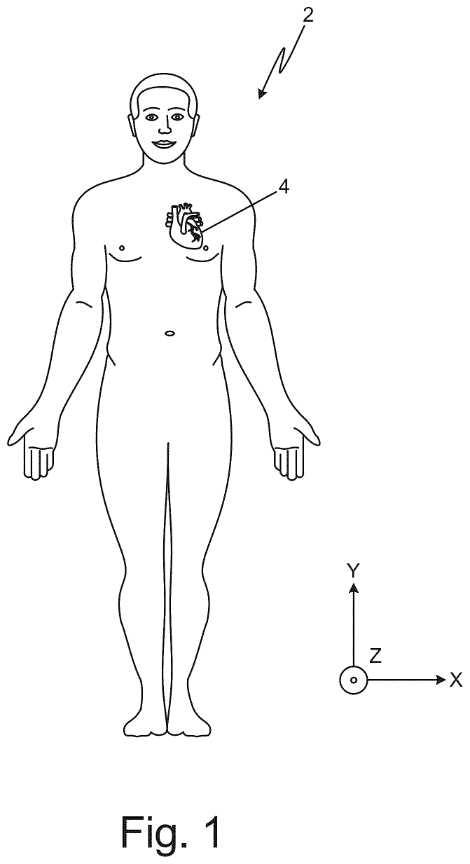

FIG. 1 is an anterior view of human patient 2 with heart 4. The body of patient 2 can generally be bisected by any of three planes: a coronal (i.e., x-y) plane, a sagittal (i.e., y-z) plane, and a transverse (i.e., x-z) plane.

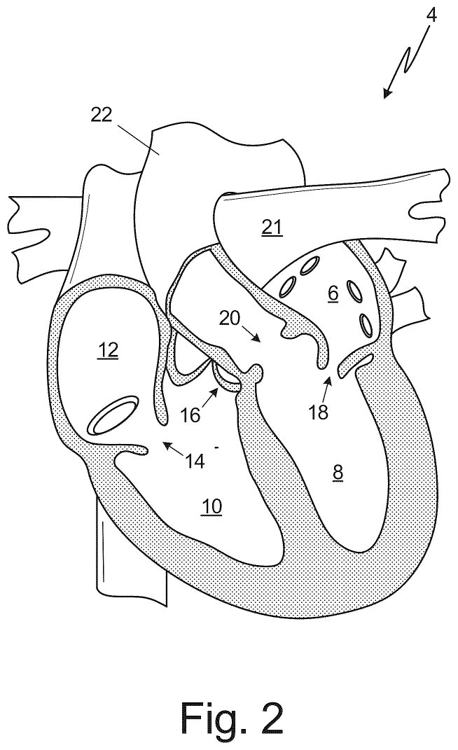

FIG. 2 is a partial cross-sectional schematic of heart 4. Heart 4 includes four chambers, including left atrium 6, left ventricle 8, right ventricle 10, and right atrium 12. The four chambers are shown in cross-section in FIG. 2. Heart 4 further includes four valves for aiding the circulation of blood therein, including tricuspid valve 14, pulmonary valve 16, mitral valve 18, and aortic valve 20. FIG. 2 further shows pulmonary artery 21 and aorta 22.

Tricuspid valve 14 separates right atrium 12 from right ventricle 10 and can include three cusps or leaflets. Tricuspid valve 14 can close during ventricular contraction (i.e., systole) and open during ventricular expansion (i.e., diastole). Pulmonary valve 16 separates right ventricle 10 from pulmonary artery 21 and may be configured to open during systole so that blood may be pumped towards the lungs, and close during diastole to prevent blood from leaking back into heart 4 from pulmonary artery 21. Similar to tricuspid valve 14, pulmonary valve 16 can have three cusps/leaflets, each one resembling a crescent. Mitral valve 18 separates left atrium 6 from left ventricle 8 and can have two cusps or leaflets. Mitral valve 18 is configured to open during diastole so that blood in left atrium 6 can flow into left ventricle 8, and close during systole to prevent blood from leaking back into left atrium 6. Aortic valve 20 separates left ventricle 8 from aorta 22. Aortic valve 20 is configured to open during systole to allow blood leaving left ventricle 8 to enter aorta 22, and close during diastole to prevent blood from leaking back into left ventricle 8.

A heart valve can include a relatively dense fibrous ring, referred to herein as the annulus, as well as a plurality of leaflets or cusps attached to the annulus. Some valves can further include a collection of chordae tendineae and papillary muscles securing the leaflets. Generally, the size of the leaflets or cusps may be such that when the heart contracts, the resulting increased blood pressure produced within the corresponding heart chamber forces the leaflets open at least partially to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage.

Heart valve disease represents a condition in which one or more of the valves of heart 4 fails to function properly. Diseased heart valves may be categorized as stenotic, wherein the valve does not open sufficiently to allow adequate forward flow of blood through the valve, and/or incompetent, wherein the valve does not close completely, causing excessive backward flow of blood through the valve when the valve is closed. In certain conditions, valve disease can be severely debilitating and even fatal if left untreated.

To treat disease of, for example, mitral valve 18, a prosthetic heart valve can be implanted in and sutured to the annulus of mitral valve 18. Such a prosthetic heart valve can be positioned with its openings oriented in the direction of blood flow from left atrium 6 to left ventricle 8. The prosthetic heart valve can be configured to operate as aortic valve 20 such that it can allow unidirectional blood flow left ventricle 8 from left atrium 6 while preventing flow in the reverse direction.

In a typical cardiac implant procedure, the heart can be incised, and in a valve replacement operation, the defective valve can be removed leaving the desired placement site that can include the valve annulus. Sutures can be passed through fibrous tissue of the annulus or desired placement site to form an array of sutures. Free ends of the sutures may be individually threaded through a suture-permeable sealing edge of the prosthetic heart valve. Artificial heart valves can be used to replace faulty or deteriorating natural heart valves in patients with heart valve disorders including aortic stenosis, mitral regurgitation, etc. The valve replacement process generally involves surgical or transcatheter procedures (e.g., balloon valvotomy) to replace the existing valves with the new artificial valves. Since the artificial valves are a foreign body, many different challenges and issues can be involved with such a procedure. For example, paravalvular leakage (PVL) and/or leaflet thickening can occur in patients who undergo heart valve replacement. Similarly, rejection of an artificial surgical heart valve due to thrombus can occur, requiring the patient to use anti-coagulants for proper valve operation.

Some methods for monitoring valve performance after implantation involve using complex bio-imaging techniques, such as echocardiography. Such methods can generally only be performed in specialized medical facilities and can cost significant time and money. Hence, such methods may generally only be used once symptoms of valve malfunction are detected. Some artificial valves may not provide an ability to detect changes in operation to detect problems early on. Moreover, many patients who suffer from valvular disease and require an artificial valve may also suffer from other cardiovascular disorders, including heart failure. Some artificial heart valve systems may not allow for gathering data about the valve and/or the patient's condition postoperatively in an outpatient setting (e.g., a cardiologist visit in a ward) using existing patient monitoring systems. Such systems may not provide for routine collection of data at sufficient resolution to enable development of new digital solutions for better management of the patients as their numbers and diversity increase over time.

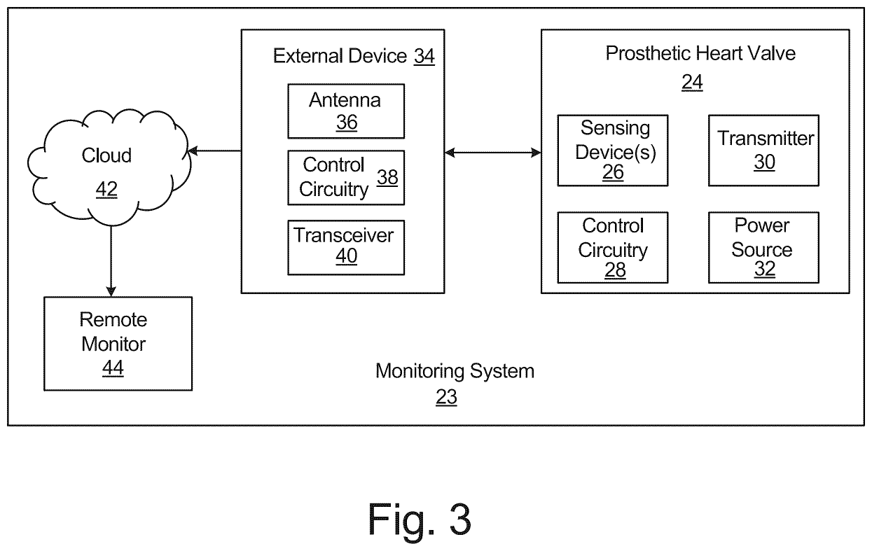

Accordingly, a prosthetic heart valve can be part of a larger system for post-operatively monitoring a patient, as will be discussed in reference to FIG. 3.

FIG. 3 is a block diagram representing monitoring system 23 for monitoring one or more physiological parameters associated with a patient (e.g., patient 2 shown in FIG. 1). System 23 includes prosthetic heart valve 24, which includes sensing devices 26, control circuitry 28, transmitter 30, and power source 32. System 23 further includes external device 34, which includes antenna 36, control circuitry 38, and transceiver 40. System 23 also includes cloud 42 and remote monitor 44.

Prosthetic heart valve 24 can include one or more sensing devices 26, control circuitry 28, transmitter 30, and power source 32. Sensing devices 28 can include one or more of following types of sensors/transducers: MEMS sensors, optical sensors, piezoelectric sensors, electromagnetic sensors, strain sensors/gauges, accelerometers, gyroscopes, and/or other types of sensors, which can be positioned in the patient to sense one or more parameters relevant to the health of the patient. Control circuitry 28 can be wired or wirelessly connected to sensing devices 26 and can include one or more of application-specific integrated circuit (ASIC), microcontrollers, chips, tuning capacitors, etc. Control circuitry 28 can receive signals from external device 34 (e.g., requests for stored or immediately acquired data), request data from sensors 26, and coordinate data transmission. Transmitter 30 can be, for example, an antenna for radiating an electronic signal transmitted by control circuitry 28. Power source 32 can be a suitable source of power able to minimize interference with the heart or other anatomy of the patient. In one example, power source 32 can be a passive means for wirelessly receiving external power (e.g., short-range or near-field wireless power transmission). In another example, power source 32 can be a battery, or a means for locally harvesting energy from within the patient.

External device 34, located at least partially outside of the patient, can be in wireless communication with prosthetic heart valve 24. External device 34 includes antenna 36, control circuitry 38, and transceiver 40. Antenna 36 can receive wireless signal transmissions from prosthetic heart valve 24. In one example, antenna 36 can be externally mounted to external device 34. Control circuitry 38 can be a processor or other suitable means for processing signals received from prosthetic heart valve 24. Transceiver 40 can be configured to receive and amplify signals from prosthetic heart valve 24, as well as to transmit signals to cloud 42 and remote monitor 44. Such signals can include, for example, pressure data acquired from sensors 26. Transceiver 40 can, accordingly, include one or more of digital-to-analog converter (DAC) circuitry, power amplifiers, low-pass filters, antenna switch modules, antennas, etc. for treatment and/or processing of transmitted and received signals.

External device 34 can serve as an intermediate communication device between prosthetic heart valve 24 and remote monitor 44. External device 34 can be a dedicated external unit designed to communicate with prosthetic heart valve 24. For example, external device 34 can be a wearable communication device, or other device that can be readily disposed in proximity to the patient and/or prosthetic heart valve 24. External device 34 can be configured to interrogate prosthetic heart valve continuously, periodically, or sporadically 24 in order to extract or request sensor-based information therefrom. In some examples, external device 34 can include a user interface upon which a user (e.g., the patient) can view sensor data, request sensor data, or otherwise interact with external device 34 and/or prosthetic heart valve 24.

Cloud 42 can be a secure network in communication with external device 34 via ethernet, Wi-Fi, or other network protocol. Cloud 42 can also be configured to implement data storage. In another example, cloud 42 can instead be a secure physical network. Remote monitor 44 can be in communication with external device 34 via cloud 42. Remote monitor 44 can be any type of computing device or collection of computing devices configured to receive, process and/or present monitor data received via cloud 42 from external device 34 or prosthetic heart valve 24. For example, remote monitor 44 can advantageously be operated and/or controlled by a healthcare entity, such as a hospital, doctor, or other care entity associated with the patient. Although certain examples disclosed herein describe communication with remote monitor 44 from prosthetic heart valve 24 indirectly through external device 34, prosthetic heart valve 24 can instead include a transmitter (e.g., transmitter 30) capable of communicating, via cloud 42, with remote monitor 44 without the necessity of relaying information through device 34.

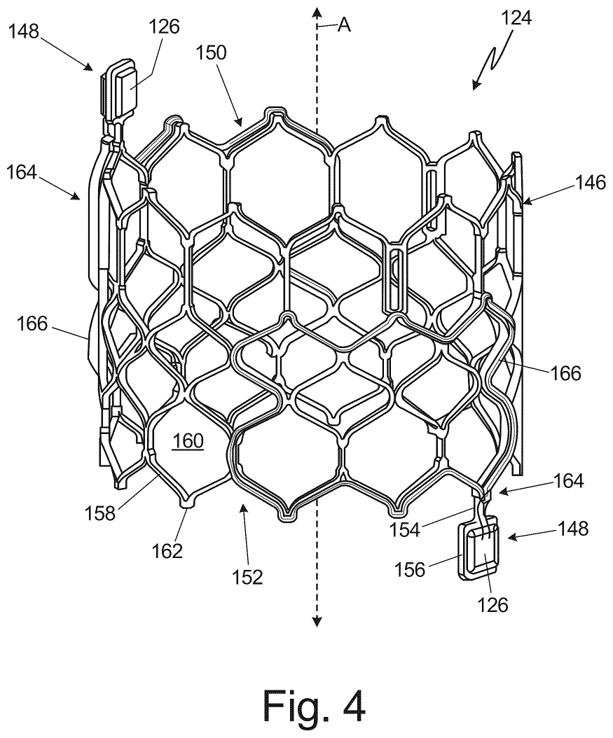

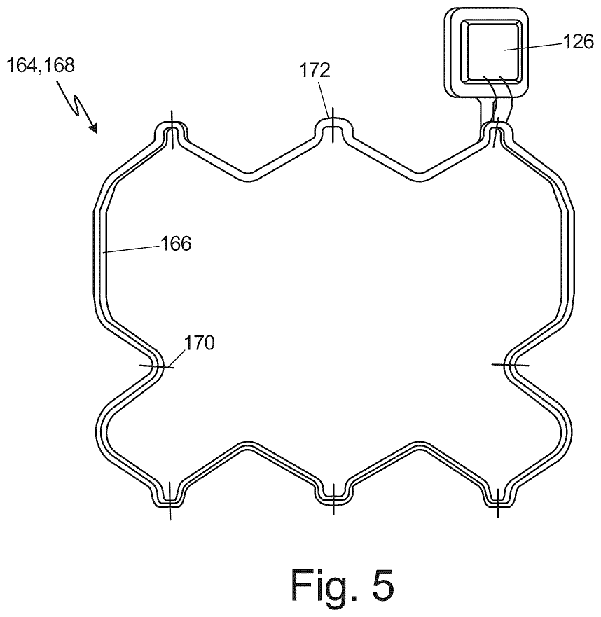

FIG. 4 is a perspective view of prosthetic heart valve 124 shown in an expanded state. FIG. 5 is a front view of a sensing circuit of prosthetic heart valve 124, shown in isolation from the frame. FIGS. 4 and 5 are discussed together.

As shown in FIG. 4, structural components of prosthetic heart valve 124 include deformable frame 146 and post assemblies 148 extending axially away from frame 146 relative to valve axis A. Axis A can generally be aligned with the direction of blood flow through prosthetic heart valve 124 when implanted in heart 4. Frame 146 can be formed from a biocompatible metallic material. As shown in FIG. 4, one post assembly 148 extends from each of top/upper end 150 and bottom/lower end 152 of prosthetic heart valve 124 based on the orientation of FIG. 4. Each post assembly 148 can include post 154 and islet 156 upon which sensor 126 can be mounted. As shown in FIG. 4, islet 156 can have a generally square shape corresponding to the shape of sensor 126. Frame 146 comprises a network of struts 158 defining open cells 160 therebetween. Each cell 160 can include oppositely axially disposed pointed tips/ends 162.

Electrical components of prosthetic heart valve 124 include one or more sensing circuits 164 for monitoring physiological parameters of patient 2. Each sensing circuit 164 includes deformable inductor coil 166 and sensor 126 electrically connected (e.g., via leads/wires) to inductor coil 166. Sensing circuit 164 can be an inductor-resistor-capacitor (LCR) circuit 168, with inductor coil 166 forming the inductor (L) and resistor (R) elements of circuit 168, and sensor 126, connected in parallel, forming the capacitor (C) element. Each LCR circuit 168 of prosthetic heart valve 126 has a distinct self-resonant frequency. The self-resonant frequency for each circuit can be represented as f=1/2π√LC(p), where L is the inductance of inductor coil 166 and C(p) is the capacitance of sensor 126 at a given pressure. In general, the self-resonant frequency for each LCR circuit 168 can range from 5 MHz to 50 MHz, and more specifically, from 10 MHz to 20 MHZ.

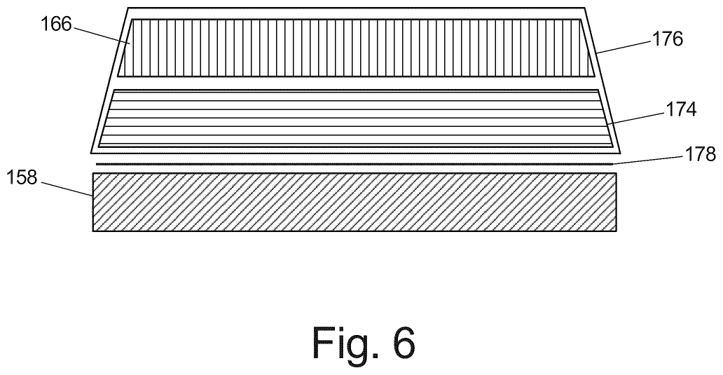

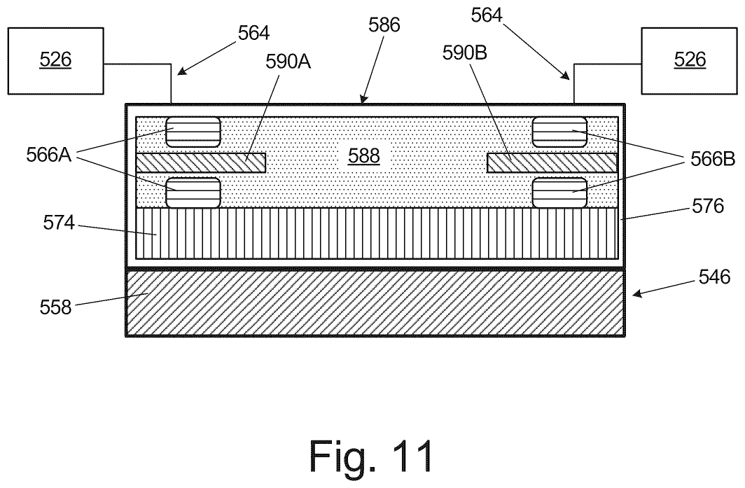

Inductor coil 166 can include one or more individual wires formed from a conductive, but biocompatible, metallic material, such as gold. Other examples can include copper or titanium. Inductor coil 166 can further be coated with an insulating coating (shown and labeled in FIG. 6). Sensors 126 can be capacitive pressure sensors in one example, each including a diaphragm and pressure cavity to form a variable capacitor to detect strain due to pressure applied to the diaphragm. In general, the capacitance of sensors 126 decreases as pressure deforms the diaphragms. To manage detuning of sensing circuit 164, a detuning mitigation layer, discussed in greater detail below with respect to FIGS. 6 and 11, can be positioned between inductor coil 166 and struts 158 of frame 146.

Inductor coil 166 can be removably attached to frame 146 by sutures 170, shown schematically in FIG. 5. Sutures 170 can be formed from a biocompatible polymer in one example. More specifically, inductor coil 166 can be attached to frame 146 in such manner as to trace a subset of struts 158 and outline a subset of cells 160. In this regard, inductor coil 166 can have nearly identical geometric attributes to struts 158 and cells 160, for example, having pointed tips 172 corresponding to pointed tips 162 of the underlying cells 160 of frame 146. In the example of FIGS. 4 and 5, inductor coil 166 can be disposed to trace/frame/outline a two by three subset (i.e., two cells high in the axial direction and three cells long in a radial dimension) of cells 160 of frame 146. This can include uppermost or lowermost cells, along with interior cells 160. Other arrangements are contemplated herein. Sutures 170 can be disposed at various points along inductor coil 166 to ensure that inductor coil 166 is secured to and maintains the shape of the supporting subset of struts 158. Suture points can include pointed tips 162 of cells 160 of frame 146 and pointed tips 172 of inductor coil 166, respectively. Additional and/or alternative suture points are contemplated herein.

FIG. 6 is a schematic cross-sectional exploded view of strut 158 and inductor coil 166 of the prosthetic heart valve of FIG. 4 taken transverse to axis A of FIG. 4. FIG. 6 shows strut 158, inductor coil 166, ferrite layer 174, insulating layer 176, and adhesive backing layer 178.

As shown in FIG. 6, strut 158 is the innermost layer, and inductor coil 166 is the outermost layer. Ferrite layer 174 is disposed between strut 158 and inductor coil 166. Ferrite layer 174 can be formed as a strip of a soft, ferromagnetic material in one example, and can mitigate detuning caused by the proximity of inductor coil 166 to metallic strut 158. Ferrite layer 174 can additionally improve (e.g., increase) the sensing range of sensing circuit 164 by shielding inductor coil 166 from magnetic field interference, induced eddy currents, etc. caused generally by electronic components of prosthetic heart valve 124. Ferrite layer 174 is ideally coextensive with inductor coil 166 such that no area of inductor coil 166 is exposed to the underlying metallic strut(s) 158. Additionally, the ferrite layer 174 should be sufficiently flexible to transition between crimped and expanded states of prosthetic heart valve 124 and maintain its position between struts 158 and inductor coil 166. Insulating layer 176 surrounds inductor coil 166 and ferrite layer 174. Ferrite layer 174 and inductor coil 166 can be in direct physical contact, (e.g., as an integrated layer) and insulating layer 176 can encircle/surround inductor coil 166 and ferrite layer 174. In one example, insulating layer 176 can be a biocompatible elastomer (e.g., silicone) or polymer (e.g., parylene or polyimide). In some examples, adhesive backing layer 178 can be included between insulating layer 176 and strut 158. Such an adhesive layer can be both biocompatible and non-conductive.

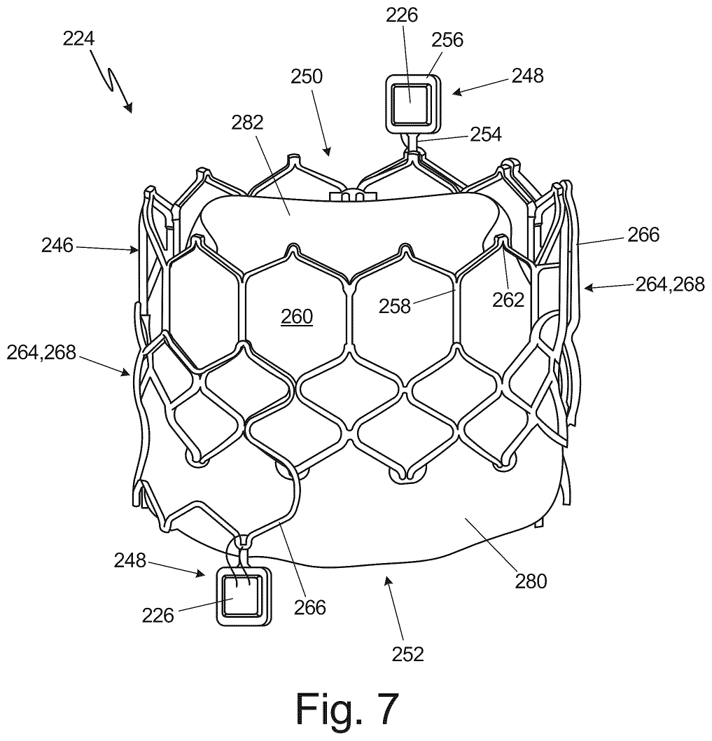

FIG. 7 is a perspective view of prosthetic heart valve 224 shown in an expanded state. Prosthetic heart valve 224 is substantially similar to prosthetic heart valve 124 shown in FIG. 4, having deformable frame 246 and post assemblies 248 extending axially away from frame 246 at each of upper end 250 and lower end 252. Post assembly 248 includes post 254 and islet 256 for mounting sensor 226 thereupon. Struts 258 of frame 246 define cells 260. Cells 260 can include oppositely disposed pointed tips 262. Prosthetic heart valve 224 further includes two sensing circuits 264, configured as LCR circuits 268, each including sensor 226 and inductor coil 266 tracing a subset of struts 258 and outlining a subset of cells 260. Inductor coil 266 can be formed from one or more conductive (e.g., gold) wires and include an underlying ferrite layer (not shown in FIG. 7). Sutures (not shown in FIG. 7) can secure each inductor coil 266 to frame 246 in the manner discussed above with respect to FIGS. 4 and 5.

Unlike the foregoing example, prosthetic heart valve 224 includes biocompatible fabric 280, configured as a skirt and partially covering frame 246. Fabric 280 can be formed from a polymer material. In an alternative example, fabric 280 can fully cover frame 246 such that no struts 258 are exposed on the outer side of frame 246. Prosthetic heart valve 224 also includes pericardium tissue 282 which can be formed from a synthetic material or derived from a mammalian (e.g., bovine) tissue source.

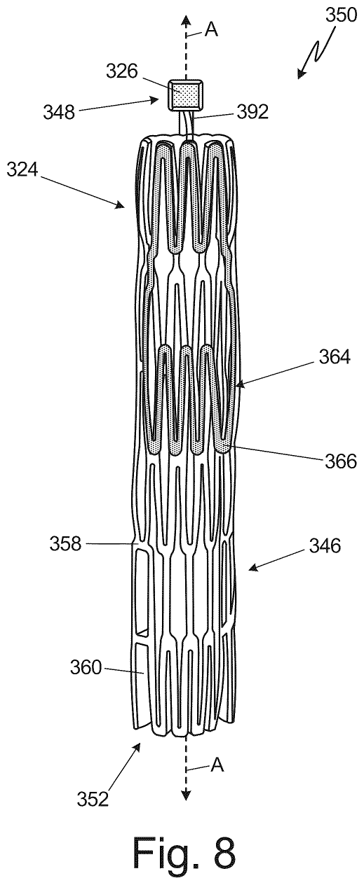

FIG. 8 is a schematic front view of prosthetic heart valve 324 shown in a crimped state. Prosthetic heart valve 324 is substantially similar to prosthetic heart valve 124 shown in FIG. 4 and prosthetic heart valve 224 shown in FIG. 7, with deformable frame 346 having interconnected struts 358 defining cells 360. Frame 346 can be formed from a biocompatible metallic material. Post assembly 348 (only one is shown in FIG. 8) extends axially away from upper end 350. Prosthetic heart valve 324 can further include at least one sensing circuit 364, with a flexible inductor coil 366 in electrical communication with sensor 326 via wire 392. In the crimped state, the axial dimension of frame 346 (i.e., along axis A) is greater than when in the expanded state, such that upper end 350 is further from lower end 352 in the crimped state. Further, in the crimped state, inductor coil 366 sutured to frame 346 will deform similarly to frame 346, maintaining the geometry of the underlying struts 358 and framing the deformed cells 360, and increasing/elongating in the axial direction. This can occur, for example, because of the relative overall flexibility of the inductor coil material and/or surrounding layers (e.g., insulating layer and detuning mitigation means), the thicknesses of the layers, and the robustness of attachment means (e.g., sutures) for securing the inductor coil to the underlying struts.

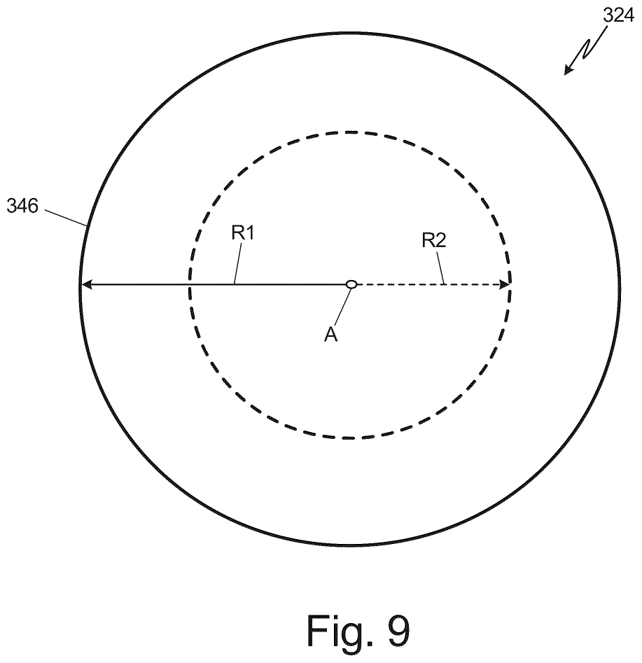

FIG. 9 is a schematic plan view comparing radial dimensions of frame 346 of prosthetic heart valve 324 in the expanded state and the crimped state. Frame 346 is the frame of prosthetic heart valve 324 shown in FIG. 8. Frame 346 in the expanded state is represented by the solid line, while frame 346 in the crimped state is represented by the dashed line. As shown in FIG. 9, in the expanded state, frame 346 has a radius R1, while in the crimped state, frame has a radius R2. R1 is greater than R2. As such, in the expanded state, the axial dimension of frame 346 is less than the axial dimension of frame 346 in the crimped state. In the expanded state, the radial dimension (i.e., radius R1) of frame 346 is greater than the radial dimension (i.e., radius R2) of frame 346 in the crimped state.

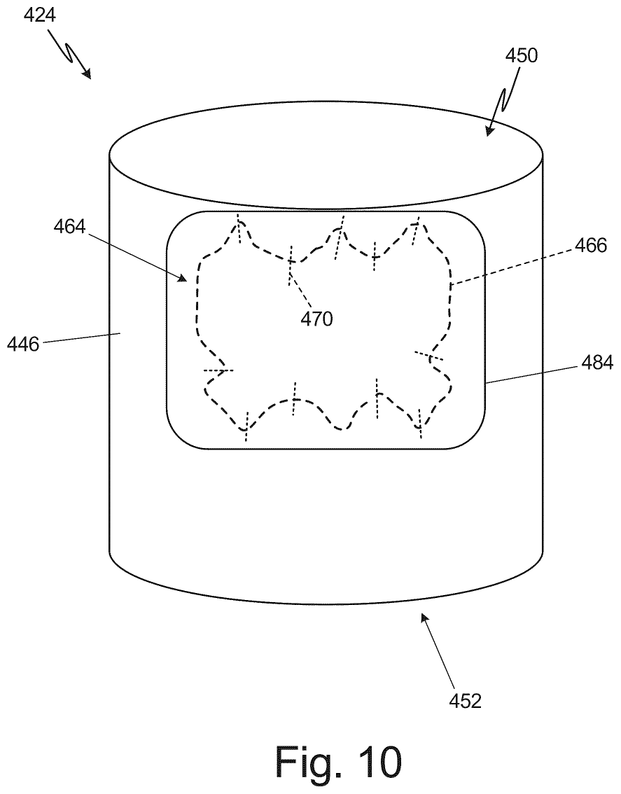

FIG. 10 is a schematic perspective view of prosthetic heart valve 424 shown in an expanded state with fabric cover 484 over inductor coil 466. For simplicity, prosthetic heart valve 424 is shown without post assemblies and sensors. Prosthetic heart valve 424 is substantially similar to prosthetic heart valve 124 shown in FIG. 4, prosthetic heart valve 224 shown in FIG. 7, and prosthetic heart valve 324 shown in FIG. 8. Prosthetic heart valve 424 has a biocompatible metallic frame 446 with interconnecting struts defining cells (not shown in FIG. 10), an upper end 450, and lower end 452. Prosthetic heart valve 424 further includes sensing circuits 464 (only one is shown) comprising inductor coil 466 and a sensor (not shown). Inductor coil 466 can be formed of one or more wires of a conductive material (e.g., gold). Sutures 470 can secure inductor coil 466 to struts of frame 446.

Prosthetic heart valves 424 differs from the previous examples in that it includes cover 484 disposed over inductor coil 466 and sutures 470. The concealed components are, accordingly, represented by dashed lines. Cover 484 can be a biocompatible fabric substantially similar to fabric 280 of FIG. 7. Cover 484 can be stitched and/or woven to frame 446 to secure it in place in one example, while in another example, cover 484 can be arranged as a sleeve circumscribing frame 446. Cover 484 can protect the concealed components from native tissue ingrowth after implantation of prosthetic heart valve 424. Cover 484 can further protect the surrounding tissues from catching on/coming into direct contact with the concealed components.

FIG. 11 is a schematic cross-sectional view of a portion of multilayer sensing assembly 586 disposed on flexible frame 546. Multilayer sensing assembly 586 incorporates multiple inductor coils 566 stacked/layered in the radial direction, relative to the axis of the encompassing prosthetic heart valve and can be used with any of the prosthetic heart valves disclosed herein.

As shown in FIG. 11, multilayer sensing assembly 586 is disposed upon metallic strut 558, which can be formed from a biocompatible metallic material. Multilayer sensing assembly 586 includes first inductor coil pair 566A and second inductor coil pair 566B, each including upper and lower coil portions printed on flexible substrate 588. Flexible substrate 588 can be formed from a polymer material, such as polyimide, and can have a thickness ranging from 2 millimeters to 3 millimeters in one example. Each inductor coil pair 566A and 566B can be printed onto flexible substrate 588, and can be formed from gold in one example, or from copper or titanium in an alternative example. Each inductor coil pair 566A and 566B can be electrically connected to a respective sensor 526, represented schematically in FIG. 11, to form sensing circuits 564. Sensors 526 can be substantially similar to sensor 126 shown in FIGS. 4 and 5, sensor 226 shown in FIG. 7, and/or sensor 326 shown in FIG. 8. Each upper coil portion of inductor coil pairs 566A and 566B can further be electrically connected (e.g., through conductive vias) to a respective lower coil portion. Inductor coil pairs 566A and 566B can be arranged to follow struts 558 and cells (not shown), having a pattern similar to inductor coil 166 shown in FIGS. 4-6, inductor coil 266 shown in FIG. 7, inductor coil 366 shown in FIG. 8, and inductor coil 466 shown in FIG. 10. Inductor coil pairs 566A and 566B can alternatively be arranged as rounded or square spirals in another example.

Multilayer sensing assembly 586 further includes flexible ferrite layer 574 disposed between strut 558 and inductor coil pairs 566A and 566B. Ferrite layer 574 can be substantially similar to ferrite layer 174 shown in FIG. 6 in that it prevents magnetic field interference and mitigates detuning of sensing circuits 564. Exemplary materials for ferrite layer 574 can include manganese-zinc (MnZn) ferrites or nickel-zinc (NiZn) ferrites. In the example shown in FIG. 11, ferrite layer 574 can be printed onto flexible substrate 588. Ferrite layer 574 can have a thickness ranging from 0.25 millimeters to 0.35 millimeters in one example. Soft magnetic layers 590A and 590B are further disposed between upper and lower coil portions of respective inductor coil pair 566A and 566B, and can be formed from frequency-dependent soft magnetic materials. More specifically, soft magnetic layer 590A can be responsive and tuned to the frequency at which inductor coil pair 566A is operating. Similarly, soft magnetic layer 590B can be responsive and tuned to the frequency at which inductor coil pair 566B is operating. Soft magnetic layers 590A and 590B thus isolate the two, different frequencies of their respective inductor coil pairs, minimizing interference and crosstalk between inductor coil pairs 566A and 566B. Ferrite layer 574, flexible substrate 588, and the various layers disposed therein can be encapsulated by insulating layer 576, which, like insulating layer 176 shown in FIG. 6, can be a biocompatible silicone, parylene, or polyimide. Insulating layer can have a thickness ranging from 50 microns to 100 microns in one example.

Implantation of any of the prosthetic heart valves discussed herein inside of a patient (e.g., patient 2 shown in FIG. 1) can include the following steps. First, a sterilized prosthetic heart valve is crimped, from an assembled expanded state, using an appropriate crimping tool so that the prosthetic heart valve can be inserted into the delivery vehicle (e.g., expandable catheter). The crimped prosthetic heart valve can be inserted into the delivery site (e.g., mitral valve 18 shown in FIG. 2), and once properly positioned, can be re-expanded and sutured to surrounding tissue. In one example, the prosthetic heart valve can be oriented such that one sensor circuit is oriented along the coronal plane to face outward from the chest. Alternatively, a sensing circuit could be oriented along the sagittal plane such that it faces the left axilla of the patient. The dimensions of the pre-crimping (i.e., assembled) expanded state and the final (i.e., re-expanded) expanded state of the prosthetic heart valve can be substantially similar in one example. In an alternative example, the final expanded state of the prosthetic heart valve can be different (e.g., smaller) than the pre-crimping expanded state. Further, the deformable nature of the inductor coils of the various examples allows for crimping and re-expansion of the prosthetic heart valve with little to no change in self-resonance and no observed diminution of circuit performance.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Discussion of Possible Examples

The following are non-exclusive descriptions of possible examples of the present invention.

A prosthetic valve includes a flexible frame disposed along a frame axis and deformable about the frame axis between a crimped state and an expanded state. The frame includes a first end, a second end oppositely disposed from the first end, and a network of interconnected struts defining a plurality of cells. The prosthetic valve further includes a first circuit mounted on the frame. The first circuit includes a first inductor coil attached to and tracing a first subset of the struts such that the first inductor coil outlines a first subset of the plurality of cells, and a first sensor in electrical communication with the first inductor coil. The first sensor is configured to sense a physical parameter and generate a signal representing the physical parameter.

The prosthetic valve of the preceding paragraph can optionally include, additionally and/or alternatively, any one or more of the following features, configurations, and/or additional components:

The prosthetic valve further includes a second circuit mounted on the frame. The second circuit includes a second inductor coil attached to and tracing a second subset of the struts such that the second inductor coil outlines a second subset of the plurality of cells, and a second sensor in electrical communication with the second inductor coil. The second sensor is configured to sense the physical parameter and generate a signal representing the physical parameter.

The prosthetic valve further includes at least a first post assembly extending axially away from the first end, and at least a second post assembly extending axially away from the second end. The first sensor is mounted on the first post assembly, and the second sensor is mounted on the second post assembly.

The first post assembly comprises a first post and a first islet, the second post assembly comprises a second post and a second islet, the first sensor is mounted to the first islet, and the second sensor is mounted to the second islet.

The prosthetic valve further includes a first detuning mitigation layer disposed between the first inductor coil and the frame, and a second detuning mitigation layer disposed between the second inductor coil and the frame.

Each of the first and second detuning mitigation layers comprise ferrite.

The prosthetic valve further includes a first insulating layer surrounding the first detuning mitigation layer and the first inductor coil, and a second insulating layer surrounding the second detuning mitigation layer and the second inductor coil.

The first and second insulating layers comprise one of silicone, parylene, and polyimide.

Each of the plurality of cells has a pointed tip.

The first inductor coil is attached to the first subset of struts at the pointed tips of each of the first subset of the plurality of cells.

The first inductor coil is removably attached to the pointed tips of each of the first subset of the plurality of cells by a plurality of sutures.

Each of the plurality of sutures are formed from a biocompatible polymer material.

The second inductor coil is attached to the second subset of struts at the pointed tips of each of the second subset of the plurality of cells.

The second inductor coil is removably attached to the pointed tips of each of the second subset of the plurality of cells by a plurality of sutures.

Each of the plurality of sutures are formed from a biocompatible polymer material.

The first subset of the plurality of cells outlined by the first inductor coil comprises two cells in an axial direction, and three cells in a radial dimension.

The second subset of the plurality of cells outlined by the second inductor coil comprises two cells in the axial direction, and three cells in the radial dimension.

The frame is formed from a biocompatible metallic material.

Each of the first and second inductor coils are formed from gold.

Each of the first and second sensors are capacitive pressure sensors, and wherein the sensed physical parameter is pressure.

The first circuit has first self-resonant frequency ranging from 5 MHz to 50 MHz.

The second circuit has a second self-resonant frequency ranging from 5 MHz to 50 MHz, the second self-resonant frequency being different from the first self-resonant frequency.

The first circuit has first self-resonant frequency ranging from 10 MHz to 20 MHz.

The second circuit has a second self-resonant frequency ranging from 10 MHz to 20 MHz, the second self-resonant frequency being different from the first self-resonant frequency.

In the crimped state, the frame has a first axial dimension and a first radial dimension, and wherein in the expanded state, the frame has a second axial dimension and a second radial dimension.

The first axial dimension is greater than the second axial dimension, and the first radial dimension is smaller than the second radial dimension.

The frame is at least partially covered with a first biocompatible fabric, the first biocompatible fabric being disposed between at least a portion of the first inductor coil or the second inductor coil and the frame.

A second biocompatible fabric covers at least one of the first inductor coil or the second inductor coil.

The prosthetic valve is implantable in a mitral valve of a patient.

In an implanted state of the prosthetic valve, the frame axis aligns with a flow of blood through the prosthetic valve.

The prosthetic valve is deliverable to the mitral valve of the patient via an expandable catheter.

The prosthetic valve is sterilized.

A method of implanting the prosthetic valve includes transitioning the prosthetic valve from the expanded state to the crimped state, delivering, via an expandable catheter, the prosthetic valve into the organ while in the crimped state, and returning the prosthetic valve to the expanded state once inside the organ of the patient.

The method of the preceding paragraph can optionally include, additionally and/or alternatively, any one or more of the following features, configurations, and/or additional components:

In the crimped state, the frame has a first axial dimension and a first radial dimension.

In the expanded state, the frame has a second axial dimension and a second radial dimension.

The first axial dimension is greater than the second axial dimension, and the first radial dimension is smaller than the second radial dimension.

The organ is a heart.

The method further includes sterilizing the prosthetic valve prior to delivering the prosthetic valve to the organ.

A prosthetic valve assembly includes a prosthetic valve that includes a flexible frame disposed along a frame axis and deformable about the frame axis between a crimped state and an expanded state. The frame includes a first end, a second end oppositely disposed from the first end, and a network of interconnected struts defining a plurality of cells. The prosthetic valve further includes a first circuit mounted on the frame. The first circuit includes a first inductor coil attached to and tracing a first subset of the struts such that the first inductor coil outlines a first subset of the plurality of cells, and a first sensor in electrical communication with the first inductor coil. The first sensor is configured to sense a physical parameter and generate a signal representing the physical parameter. The prosthetic valve assembly further includes a transmitter in communication with the first sensor.

The prosthetic valve assembly of the preceding paragraph can optionally include, additionally and/or alternatively, any one or more of the following features, configurations, and/or additional components:

The prosthetic valve further includes a second circuit mounted on the frame. The second circuit includes a second inductor coil attached to and tracing a second subset of the struts such that the second inductor coil outlines a second subset of the plurality of cells, and a second sensor in electrical communication with the second inductor coil. The second sensor is configured to sense the physical parameter and generate a signal representing the physical parameter.

The prosthetic valve further includes at least a first post assembly extending axially away from the first end, and at least a second post assembly extending axially away from the second end. The first sensor is mounted on the first post assembly, and the second sensor is mounted on the second post assembly.

The transmitter is in communication with the second sensor.

The prosthetic valve assembly further includes a power source in wired or wireless communication with the prosthetic valve assembly.

A monitoring system includes the prosthetic valve assembly and an external device in communication with the prosthetic valve assembly.

The monitoring system of the preceding paragraph can optionally include, additionally and/or alternatively, any one or more of the following features, configurations, and/or additional components:

The external device comprises a transceiver in wireless communication with the prosthetic heart valve assembly.

The monitoring system further includes a remote monitor in communication with the prosthetic heart valve assembly via the external device.

A prosthetic valve includes a flexible frame disposed along a frame axis and deformable about the frame axis between a crimped state and an expanded state. The frame includes a first end, a second end oppositely disposed from the first end, and a network of interconnected struts defining a plurality of cells. The prosthetic valve further includes a multilayer sensing assembly mounted on the frame. The multilayer sensing assembly includes a first inductor coil pair comprising first upper and lower inductor coil portions, the first inductor coil pair disposed on a flexible substrate, a detuning mitigation layer disposed between the frame and the flexible substrate, and a first sensor in electrical communication with the first inductor coil pair. The first sensor is configured to sense a physical parameter and generate a signal representing the physical parameter.

The prosthetic valve of the preceding paragraph can optionally include, additionally and/or alternatively, any one or more of the following features, configurations, and/or additional components:

The multilayer sensing assembly further includes a second inductor coil pair comprising second upper and lower inductor coil portions, the second inductor coil pair disposed on the flexible substrate, and a second sensor in electrical communication with the second inductor coil pair. The second sensor is configured to sense the physical parameter and generate a signal representing the physical parameter.

The detuning mitigation layer comprises ferrite.

The prosthetic valve further includes an insulating layer encapsulating the detuning mitigation layer and the flexible substrate.

The insulating layer comprises one of silicone, parylene, and polyimide.

A thickness of the insulating layer ranges from 50 microns to 100 microns.

The flexible substrate comprises polyimide.

A thickness of the flexible substrate ranges from 2 millimeters to 3 millimeters.

A thickness of the detuning mitigation layer ranges from 0.25 millimeters to 0.35 millimeters.

The prosthetic valve further includes a first soft magnetic layer disposed on the flexible substrate between the first upper and lower inductor coil portions, and a second soft magnetic layer disposed on the flexible substrate between the second upper and lower inductor coil portions.

The frame is formed from a biocompatible metallic material.

Each of the first and second inductor coil pairs are formed from one of gold, copper, and titanium.

Each of the first and second sensors are capacitive pressure sensors, and wherein the sensed physical parameter is pressure.

The prosthetic valve is implantable in a mitral valve of a patient.

The prosthetic valve is deliverable to the mitral valve of the patient via an expandable catheter.

The prosthetic valve is sterilized.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A prosthetic valve comprising:
a flexible frame disposed along a frame axis and deformable about the frame axis between a crimped state and an expanded state, the frame comprising:
a first end;
a second end oppositely disposed from the first end; and
a network of interconnected struts defining a plurality of cells;
a first circuit mounted on the frame, the first circuit comprising:
a first inductor coil attached to and tracing a first subset of the struts such that the first inductor coil outlines a first subset of the plurality of cells; and
a first sensor in electrical communication with the first inductor coil;
wherein the first sensor is configured to sense a physical parameter and generate a signal representing the physical parameter.

2. The prosthetic valve of claim 1 and further comprising:
a second circuit mounted on the frame, the second circuit comprising:
a second inductor coil attached to and tracing a second subset of the struts such that the second inductor coil outlines a second subset of the plurality of cells; and
a second sensor in electrical communication with the second inductor coil;
wherein the second sensor is configured to sense the physical parameter and generate a signal representing the physical parameter.

3. The prosthetic valve of claim 2 and further comprising:
at least a first post assembly extending axially away from the first end; and
at least a second post assembly extending axially away from the second end;
wherein the first sensor is mounted on the first post assembly; and
wherein the second sensor is mounted on the second post assembly.

4. The prosthetic valve of claim 3, wherein:
the first post assembly comprises a first post and a first islet;
the second post assembly comprises a second post and a second islet;
the first sensor is mounted to the first islet; and
the second sensor is mounted to the second islet.

5. The prosthetic valve of claim 2 and further comprising:
a first detuning mitigation layer disposed between the first inductor coil and the frame; and
a second detuning mitigation layer disposed between the second inductor coil and the frame.

6. The prosthetic heart valve of claim 5, wherein each of the first and second detuning mitigation layers comprise ferrite.

7. The prosthetic heart valve of claim 6 and further comprising:
a first insulating layer surrounding the first detuning mitigation layer and the first inductor coil; and
a second insulating layer surrounding the second detuning mitigation layer and the second inductor coil.

8. The prosthetic heart valve of claim 7, wherein the first and second insulating layers comprise one of silicone, parylene, and polyimide.

9. The prosthetic valve of claim 2, wherein:
the first inductor coil is attached to the first subset of struts at pointed tips of each of the first subset of the plurality of cells;
the first inductor coil is removably attached to the pointed tips of each of the first subset of the plurality of cells by a first plurality of sutures;
the second inductor coil is attached to the second subset of struts at the pointed tips of each of the second subset of the plurality of cells; and
the second inductor coil is removably attached to the pointed tips of each of the second subset of the plurality of cells by a second plurality of sutures.

10. The prosthetic valve of claim 2, wherein the frame is formed from a biocompatible metallic material.

11. The prosthetic valve of claim 2, wherein each of the first and second inductor coils are formed from gold.

12. The prosthetic valve of claim 2, wherein each of the first and second sensors are capacitive pressure sensors, and wherein the sensed physical parameter is pressure.

13. The prosthetic valve of claim 2, wherein the first circuit has first self-resonant frequency ranging from 5 MHz to 50 MHz, and wherein the second circuit has a second self-resonant frequency ranging from 5 MHz to 50 MHZ, the second self-resonant frequency being different from the first self-resonant frequency.

14. The prosthetic valve of claim 2, wherein the frame is at least partially covered with a first biocompatible fabric, the first biocompatible fabric being disposed between at least a portion of the first inductor coil or the second inductor coil and the frame.

15. The prosthetic valve of claim 14, wherein a second biocompatible fabric covers at least one of the first inductor coil or the second inductor coil.

16. The prosthetic valve of claim 2, wherein the prosthetic valve is sterilized.

17. A monitoring system comprising:
a prosthetic valve assembly comprising:
the prosthetic valve of claim 2:
a transmitter in communication with the first sensor and the second sensor;
an external device in communication with the prosthetic valve assembly;
wherein the external device comprises a transceiver in wireless communication with the prosthetic heart valve assembly.

18. A prosthetic valve comprising:
a flexible frame disposed along a frame axis and deformable about the frame axis between a crimped state and an expanded state, the frame comprising:
a first end;
a second end oppositely disposed from the first end;
a network of interconnected struts defining a plurality of cells;
a multilayer sensing assembly mounted on the frame, the multilayer sensing assembly comprising:
a first inductor coil pair comprising first upper and lower inductor coil portions, the first inductor coil pair disposed on a flexible substrate;
a detuning mitigation layer disposed between the frame and the flexible substrate; and
a first sensor in electrical communication with the first inductor coil pair;
wherein the first sensor is configured to sense a physical parameter and generate a signal representing the physical parameter.

19. The prosthetic valve of claim 18, and further comprising:
an insulating layer encapsulating the detuning mitigation layer and the flexible substrate.

20. The prosthetic valve of claim 18, and further comprising:
a first soft magnetic layer disposed on the flexible substrate between the first upper and lower inductor coil portions.
